(19) United States
(12) Patent Application Publication
(10) Pub. No.: US 2024/0287642 A1
(43) Pub. Date: Aug. 29, 2024

(54) PROCESS

(71) Applicant: Cemvita Factory, Inc., Houston, TX (US)

(72) Inventors: Stephan Christel, Westminster, CO (US); Tom Clark, Westminster, CO (US); Marny Reakes, Westminster, CO (US); Jayme Feyhl-Buska, Westminster, CO (US); Jillian Ayers, Westminster, CO (US); Colbey Denvin, Westminster, CO (US)

(21) Appl. No.: 18/174,170

(22) Filed: Feb. 24, 2023

(51) Int. Cl. C22B 3/18; C12N 1/20

(52) U.S. Cl. CPC C22B 3/18; C12N 1/20

(57) ABSTRACT

The present application provides a process comprising: i) providing a body of metal ore, ii) contacting the body of metal ore with a microbial lixiviant comprising one or more populations of microorganisms, iii) measuring at least one parameter indicative of function of the one or more populations of microorganisms, and iv) adjusting one or more process variables in response to the measured parameter.

### FIELD

The present application provides metal leaching processes as well as compositions useful in such processes.

### BACKGROUND

The extraction of metals and minerals from the earth has been of importance to humanity for millennia. Techniques for doing so have evolved over time. In recent years, there have been growing concerns regarding the environmental impact of mining metal ores and extracting the metal of interest therefrom. Mining, tunnelling, and blasting have profound effects on the physical environment. Transport and extraction of metals from ore introduce toxic chemicals into the environment and utilize significant amounts of energy.

It is therefore not surprising that steps have been taken to minimize the environmental impact of extracting metal from ore. One such approach is leaching followed by solvent extraction/electrowinning (known as SX/EW), which has been primarily used to obtain copper and gold, as well as other metals such as uranium, zinc, and nickel.

With such leaching approaches, ore containing the mineral of interest is exposed to a lixiviant (e.g., an acid such as sulfuric acid) to leach out the metal of interest. The metal from the metal-rich leachate (or pregnant liquor solution) is then extracted from the leachate using an organic solvent (solvent extraction) and then transferred to an electrolyte. The electrolyte is then electrolyzed, forming the metal of interest in a pure form (i.e., electrowinning). Examples of leaching processes are disclosed in Australian Patent Application No. 2004240193, European Patent Publication No. 2024523 and International Patent Publication No. WO02/081761.

There are multiple ways in which such leaching approaches have been deployed in practice.

One such method of deploying the leaching approach is via dump or heap leaching processes. In these methods, ore is mined, crushed into smaller particulate form, and then amassed into a dump or heap. The dump or heap of particulate ore then has leaching lixiviant applied to it (e.g., by pouring or spraying). The lixiviant then flows through the dump or heap, leaching out the metal of interest, and the metal-rich leachate is then extracted from the base of the heap or dump.

A second example of how the leaching approach can be deployed is via tank-leaching—which is similar in approach to dump or heap leaching in that ore is firstly mined and crushed into particulate form. However, rather than a dump or heap of the particulate ore then being formed, the particulate ore is instead transferred to a tank or container containing the lixiviant. Such an approach may be preferable to dump or heap leaching where the metal of interest is particularly refractory to leaching. Additionally, the lixiviant is contained in a controlled environment meaning that the conditions in which the leaching step is performed can be optimized, and additionally the risk of inadvertent loss of solution to the environment is reduced.

One further example is in situ leaching. As the name suggests, the leaching step is conducted in situ, i.e., without having to firstly mine, tunnel, or blast out ore and then transport it for processing.

While leaching techniques have been successfully commercially deployed, attempts to improve their efficiency have been made. One such approach has been to employ lixiviants comprising bacteria, for example bacteria which are capable of metabolizing sulfur and/or iron. As those skilled in the art will be aware, minerals containing sulfur and iron are significant components of metal-containing ores. By exposing such ores to bacteria which are capable of metabolizing sulfur and/or iron, these minerals can be broken up and released into solution, thus freeing up the entrapped metal for leaching by the lixiviant.

Such ‘bioleaching’ processes have been commercially employed to extract copper and gold, and to a lesser extent uranium, zinc, and nickel. Research is also currently underway to explore the applicability of bioleaching to the extraction of rare earth elements. Thus, bioleaching has wide theoretical applicability.

Examples of bioleaching processes are known. For example, US 2008/0127779 discloses a process to increase the bioleaching speed of ores or concentrates of sulfide metal species by means of continuous inoculation with leaching solution that contains isolated microorganisms, with or without the presence of native microorganisms. US 2011/0045581 discloses a bioreactor for continuous production of bioleaching solutions for inoculation and irrigation of sulfide-ore bioleaching heaps and dumps.

However, one issue with conventional bioleaching applications is that following a period in which the microbial composition functions effectively to leach metal from the ore, a marked reduction in activity occurs. As and when a reduction in leaching efficiency is noted, fresh bacterial lixiviant is applied to attempt to maintain or revive leaching activity. While this approach can be effective, the successful revival in leaching activity is at best temporary, necessitating repeated applications of bacterial lixiviant which adds to cost and complexity. Frequently, a completed revival in leaching efficiency with only a partial restoration being observed, if at all. In order to exploit the full potential of bioleaching, there is a need for more efficient processes and/or improved lixiviant compositions.

### SUMMARY

Thus, according to a first aspect of the present application, there is provided a process comprising: i) providing a body of metal ore, ii) contacting the body of metal ore with a microbial lixiviant comprising one or more populations of microorganisms, iii) measuring at least one parameter indicative of function of the one or more populations of microorganisms, and iv) adjusting one or more process variables in response to the measured parameter.

The inventors have identified that a shortcoming of conventional bioleaching methods is that the approach of periodically replenishing bacterial lixiviant results in inefficient bioleaching performance. By monitoring the function of the microbial lixiviant through the measurement of one or more parameters, this advantageously permits optimized bioleaching performance in terms of the functional lifespan of the lixiviant and/or the amounts of metal that can be recovered.

One benefit of the process of the present invention is its versatility. The bioleaching method can be applied to different leaching scenarios. Thus, in one embodiment, the body of metal ore is a heap optionally in proximity to the site from which the ore was mined. In such embodiments, the step of contacting the ore with the microbial lixiviant may be conducted by supplying the microbial lixiviant to the heap. In embodiments, the microbial lixiviant may be supplied to the heap at one or more locations on or in the heap. Additionally or alternatively, microbial lixiviant may be supplied to the heap on its surface or within the body of the heap. Any apparatus used to supply lixiviant to heaps may be utilized.

In certain embodiments, microbial lixiviant may be supplied to the heap during its assembly, i.e. in the heap stacking phase.

Additionally or alternatively, the body of metal ore may be provided in a tank. In such embodiments, the step of contacting the metal ore with the microbial lixiviant may be conducted by supplying the microbial lixiviant into the tank.

In addition to versatility regarding the scenarios in which the process of the present application may be utilized, it may also be employed to extract a wide range of metals from ores. In embodiments of the present application, the metal ore may comprise copper. Examples of copper-containing ores which may be treated using the process of the present invention include chalcopyrite, cuprite, covellite, chalcocite, bornite, malachite, azurite, antlerite, tetrahedrite, chrysocolla, tennantite, enargite atacamite, brochantite, dioptase, rosasite, digenite, dioptase, tenorite, pseudomalachite, heterogenite, kolwezite and/or turquoise.

Additionally or alternatively, the metal ore may comprise of minerals containing gold, uranium, zinc, lead, arsenic, antimony, nickel, molybdenum, silver, and cobalt. Examples include galena, sphalerite, smithsonite, hemimorphite, zincite, willemite, hydrozincite, molybdenite, wulfenite, cobaltite, smaltite, erythrite, glaucodot, linnaeite, pentlandite, serpentine, saprolite, laterite, smectite, garnierite pentlandite, calaverite, krennerite, nagyagite, petzite and sylvanite.

The metal ore may be in a range of forms. For example, the metal ore may be provided in its native form, for example, in in situ operations, in pulverized form, in crushed form, for example as run-off mine (ROM) material, as granules, as a clay, or as a slurry.

### BRIEF DESCRIPTION OF THE FIGURES

FIG. 1. Illustrates that contaminants can accumulate, temperature can drop, and overall leaching efficiency can decrease.

FIG. 2. Demonstrates the control of the populations of microorganisms present in the microbial lixiviant is an effective and convenient way to maximize lixiviant functional lifespan and/or leaching efficiency

FIGS. 3 and 4 demonstrate that the copper leaching efficiency of a lixiviant comprising a rationally designed consortium of microorganisms belonging to different populations (iron oxidizers, sulfur oxidizers and organic carbon degraders.

FIG. 5. Shows the modelled and a decline in the rate of copper extraction with increasing concentration of organic carbon when using Inoculant A and its recovery when replacing the inoculant with Inoculant B.

### DETAILED DESCRIPTION

As explained above, the inventors have identified that through process control, the functional lifespan and/or leaching efficiency of the microbial lixiviant can be maximized. This is achieved through monitoring of one or parameters which are indicative of the function of the one or more populations of microorganisms.

In one embodiment, the method comprises making a temperature measurement. The temperature of the body of metal ore and/or the microbial lixiviant may be measured. The generation of heat by the microbial lixiviant is indicative of its function, specifically its metabolism of mineralic compounds in the ore such as recalcitrant sulfides. If a reduction of lixiviant and/or ore temperature is observed, this is indicative of a reduction in microbial function.

Measuring the temperature of the metal ore may be conducted using techniques known to those skilled in the art. For example, temperature probes may be used. In embodiments in which the body of metal ore is a heap, the temperature probe may be located on or within the heap. In embodiments in which the body of metal ore is provided in a tank, the temperature probe may be located within the tank.

As used herein, the term “heap” is to be interpreted broadly to encompass conventional leaching heaps and dumps as well as other structures of metal ore which are capable of being leached.

In certain embodiments, the method may comprise assessing metabolite levels in the lixiviant. For a given microbial population, the production of specific metabolites is indicative of the metabolic function of that population. Thus, the levels of a specific metabolite may be determined. If a reduction in the level of a specific metabolite is observed, this may be indicative of a reduction in the metabolic function of one microorganism population in the microbial lixiviant.

Additionally or alternatively, the composition of metabolites may be assessed. If the profile of metabolites present in the lixiviant is altered, this is indicative of the abundance or metabolic function of microbial populations in the lixiviant shifting which could result in the microbial lixiviant metabolizing substrates other than the substrate of interest or the generation of contaminants which can damage the metabolic function of the microbial lixiviant.

The assessment of metabolite levels and/or composition may be achieved through obtaining a sample of metal ore contacted with the lixiviant, the lixiviant itself and/or efflux from the body of ore and assessing metabolite content using routine techniques. In embodiments in which the body of metal ore is a heap, a core sample from within the heap may be taken and the metabolite content assessed. Additionally or alternatively, the metabolite content of efflux from the heap may be assessed.

In certain embodiments, the method may comprise assessing nucleic acid profiles, for example in the lixiviant and/or in the body of metal ore. In embodiments, the nucleic acid may be DNA and/or RNA. The nucleic acid may be isolated.

In embodiments of the present application, the profiles of specific nucleic acid (e.g. DNA and/or RNA) sequences may be assessed. Thus, the levels of one or more specific sequences may be assessed. In embodiments, the levels of a specific sequence may be assessed, for example because the presence of that sequence is indicative of the function and/or prevalence of a specific strain of microorganism and/or a specific population of microorganisms. In certain embodiments, the levels of a number of different specific sequences may be assessed, for example to enable a determination to be made as to whether there are fluctuations in the identity and levels of nucleic acids which may be indicative of stressed and thus sub optimally performing populations.

In embodiments of the present application, the presence, absence or levels of specific sequences of nucleic acid may be assessed, for example the coded or transcribed 16S rRNA gene, coded or transcribed genes involved in microbial iron oxidation, coded or transcribed genes involved in microbial inorganic sulfur compound oxidation, coded or transcribed genes involved in the mediation of heavy metal toxicity in microorganisms. If specific sequences of nucleic acid are identified, or if levels of those sequences are altered, this is indicative of fluctuations in microbial community composition and/or functional capabilities.

The assessment of nucleic acid levels and/or the presence, absence and or levels of specific sequences may be achieved through obtaining a sample of metal ore, the microbial lixiviant and/or efflux from the body of ore and assessing nucleic acid content using routine techniques, for example DNA or RNA extraction, amplification via quantitative PCR, and/or sequencing, e.g., using Illumina sequencing technology. In embodiments in which the body of metal ore is a heap, a core sample from within the heap may be taken and the metabolite content assessed. Additionally or alternatively, the metabolite content of efflux from the heap may be assessed.

Additionally or alternatively the method may comprise determining the abundance and/or composition of the microbial populations present in the microbial lixiviant and/or the microbial diversity of the microbial lixiviant. The assessment of the abundance of the microbial populations present in the microbial lixiviant and/or the microbial diversity of the microbial lixiviant may be achieved through obtaining a sample of metal ore contacted with the lixiviant and assessing the microbial population using techniques including sequencing tools such as high throughput meta-genomic and transcriptomic or metatranscriptomic sequencing. Where the microbial diversity of the microbial lixiviant is assessed, this may comprise be presented using the Shannon index, the Chaol index, or any other form of diversity index known to those skilled in the art. In embodiments in which the body of metal ore is a heap, a sample from within the heap and/or from its surface may be taken and the microbial composition/diversity assessed. Additionally or alternatively, the microbial composition/diversity in efflux from the heap may be assessed.

In certain embodiments, a pH measurement may be made. Variations in pH may be indicative of changes in the abundance of populations of microbes in the lixiviant. For example, an increase in the proportion of sulfur oxidizing microbes may result in the release of sulfuric acid causing a decrease in lixiviant pH.

pH may be measured using techniques known to those skilled in the art. In embodiments, pH may be measured by taking a sample of lixiviant and/or metal ore and assessing the pH. pH of metal ore in solid form may be assessed by firstly mixing the ore with a liquid medium, e.g. water, and then measuring pH. The sample of metal ore may be taken from within the body of metal ore and/or from the surface thereof. In embodiments (e.g. in which the body of metal ore is provided as a heap), the sample may be collected in efflux from the heap. Standard pH measurement apparatus may be employed, for example, a conventional pH meter.

In embodiments of the present application, organic carbon content levels may be measured. At initiation of a bioleaching process, organic carbon content is typically low, but can accumulate or increase over time due to inclusions of soil or clay in the ore body, introduction of foreign organic matter during ore processing, autotrophic microbial growth, and carryover from solvent extraction chemicals. Microorganisms which may be employed in bioleaching processes which effectively catalyse mineral dissolution may be sensitive to the inhibitory effects of organic carbon, for example at low pH. As the bioleaching process progresses, the populations present in the microbial lixiviant may be adjusted depending on the measured organic carbon.

In such embodiments, organic carbon content may be measured by taking a sample of lixiviant and/or metal ore and assessing the organic carbon content. The sample of metal ore may be taken from within the body of metal ore and/or from the surface thereof. In embodiments (e.g. in which the body of metal ore is provided as a heap), the sample may be collected in efflux from the heap.

In some embodiments, the process may comprise measuring the levels of ore-derived toxins. Variations in the levels of such toxins may require adjustment of microbial populations which are more robust toward specific toxins and/or which are capable of metabolising the toxin or rendering it less toxic to the microbial lixiviant in some other way, for example via sequestration or precipitation. As an example, metal ore bodies can contain fluoride at varying concentrations. Stacking of ore with increasing concentrations of fluoride can result in an increase in the concentration of fluoride in process fluids and in the heap. This increase in fluoride levels may negatively affect the function of microbial population. Thus, if an increase in fluoride levels is measured, the composition of the microbial lixiviant supplied to the heap may be adjusted to comprise alternative microbe populations with higher tolerance to fluoride.

Those skilled in the art will be familiar with compounds that may be released from metal ores in bioleaching processes which are toxic to microbes in lixiviant compositions. Examples of these include arsenic, halides including fluoride, lead and silver.

In such embodiments, ore-derived toxin content may be measured by taking a sample of lixiviant and/or metal ore and assessing the ore-derived toxin content. The sample of metal ore may be taken from within the body of metal ore and/or from the surface thereof. In embodiments (e.g. in which the body of metal ore is provided as a heap), the sample may be collected in efflux from the heap.

In certain embodiments, Oxidation Reduction Potential (ORP) may be assessed. Copper primary minerals are often recalcitrant, but have been observed to be amenable to leaching at low ORP. As such, ORP can be used as a measure of leaching efficiency. As ORP in sulfide leaching systems are determined by the ratio of Fe3+/Fe2+, modulation of populations of iron oxidizing microbes can be used to decrease ORP and thus increase leaching efficiency.

In embodiments, one or more parameters indicative of function of the microbial lixiviant, such as those detailed above, may be measured. In certain embodiments, 2 or more such parameters may be measured. In embodiments, 3 or more such parameters may be measured. In certain embodiments, 4 or more such parameters may be measured. In some embodiments, 5 or more such parameters may be measured.

In the process of the present application, at least two measurements of a given parameter may be made. In such embodiments, one may be a baseline measurement, e.g. made at or around the time that the microbial lixiviant is contacted with the metal ore, with one or more subsequent measurements of that parameter being made following contacting of the metal ore with the microbial lixiviant. In such embodiments, a comparison may be made between the baseline measurement and the one or more subsequent measurements to determine whether there has been any change in the measured parameter.

In embodiments of the present application, steps iii) and iv) of the process of the present application may be repeated (optionally multiple times), i.e. such that multiple assessments of metabolic function of the microbial lixiviant are made and multiple interventions are carried out to ensure optimal leaching efficiency. In certain embodiments, one or more parameters indicative of function of the one or more populations of microorganisms may be continuously measured or may be intermittently measured. For the avoidance of doubt, it is not essential in the process of the present application that when a parameter is measured in step iii) of that process, an adjustment of one or more process variables must necessarily be made in response to the measured parameter; multiple measurements of the parameter may be made before an adjustment is made to the one or more process variables.

In embodiments, measurements of at least one parameter indicative of function of the one or more populations of microorganisms may be taken at different locations. For example, if the metal ore is provided as a heap, then measurements of one or more parameters indicative of function of the one or more populations of microorganisms may be taken at different locations within the heap.

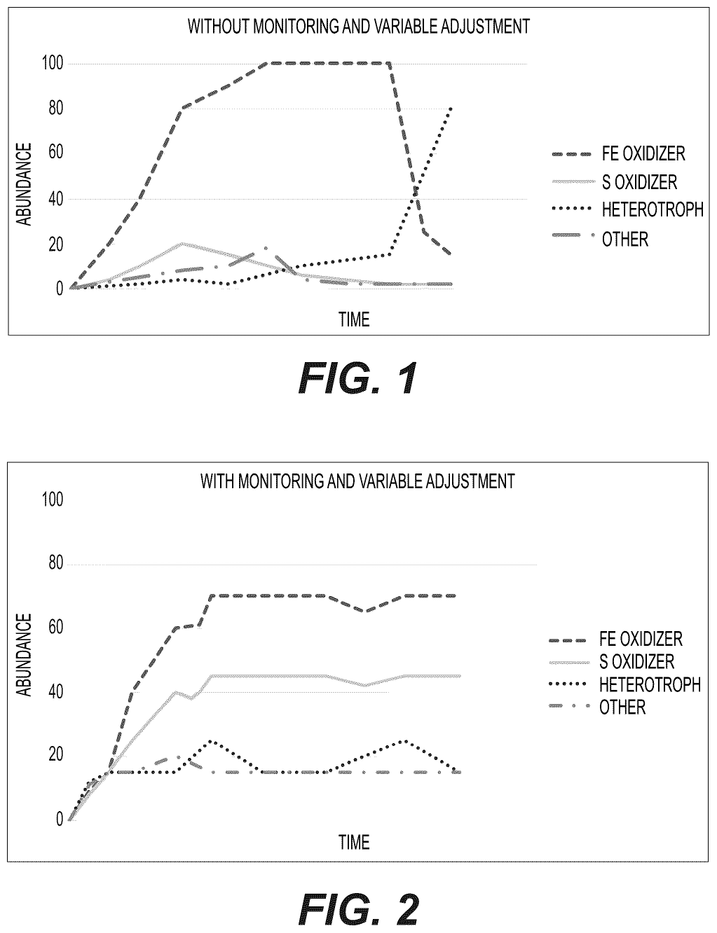

The inventors have found that the microbial diversity of a body of metal ore being subjected to a bioleaching process changes over time and while the total biomass present may be high, depending on the duration over which that biomass has been in contact with the body of metal ore, the biodiversity of the biomass can be depleted. As a consequence, contaminants can accumulate, temperature can drop, and overall leaching efficiency can decrease. This is graphically depicted in FIG. 1.

Thus, the inventors have recognized that the control of the populations of microorganisms present in the microbial lixiviant is an effective and convenient way to maximize lixiviant functional lifespan and/or leaching efficiency as depicted in FIG. 2. By doing so, lower levels of biomass can be supplied to bodies of metal ore such as heaps than in conventional processes where the aim was simply to maximise the biomass present in the heap on the assumption that this would maximise leaching function.

Thus, according to a further aspect of the present invention, there is provided a microbial lixiviant composition for use in a bioleaching process comprising a plurality of strains of microorganisms forming one or more populations of microorganisms selected from the following: iron oxidizers, sulfur oxidizers, organic carbon degraders and/or toxin inactivating microorganisms. Such compositions may be employed in the process of the present invention.

The microbial lixiviant may comprise bacteria, archaea, fungi, algae or other types of microorganism. Examples of genera of microorganisms which may be employed in the microbial lixiviants discussed herein include, but not limited to I. Iron-oxidizers: Acidocella, Acidimicrobium (e.g. Acidimicrobium ferrooxidans), Acidiphilium, Acidiplasma, Acidithiobacillus (e.g. Acidithiobacillus ferrivorans, Acidithiobacillus ferrooxidans), Acidithiomicrobium, Acidithrix (e.g. Acidithrix ferrooxidans), Ferrithrix (e.g. Ferrithrix thermotolerans), Ferroplasma, Ferrovum (e.g. Ferrovum myxofaciens), Halomonas, Leptospirillum (e.g. Leptospirillum ferrooxidans, Leptospirillum ferriphilum), Metallosphaera, Mycobacterium, Sulfobacillus (e.g. Sulfobacillus thermosulfidooxidans), Sulfolobus (e.g. Sulfolobus metallicus) and Thermogymnomonas. II. Sulfur oxidizers: Acidianus, Acidicaldus (e.g. Acidicaldus organivorans), Acidithiobacillus (e.g. Acidithiobacillus caldus), Ferviacidithiobacillus, Igneacidithiobacillus, Metallosphaera and Sulfolobus (e.g. Sulfolobus acidocaldarius). III. Organic carbon degraders: Acidicaldus (e.g. Acidicaldus organivorans), Acidianus, Acidocella, Acidimicrobium (e.g. Acidimicrobium ferrooxidans), Acidiphilium, Acidiplasma, Acidimicrobium (e.g. Acidimicrobium ferrooxidans), Acidithrix (e.g. Acidithrix ferrooxidans), Alicyclobacillus (e.g. Alicyclobacillus disulfoxidans), Ferrithrix (e.g. Ferrithrix thermotolerans), Ferroplasma, Ferrovum (e.g. Ferrovum myxofaciens), Metallosphaera, Picrophilus, Sulfobacillus (e.g. Sulfobacillus thermosulfidooxidans), Sulfolobus and Thermoplasma.

A population that may be provided in the microbial lixiviant are toxin inactivating microorganisms. These organisms have the function of lowering the concentration of toxins in the microbial lixiviant and/or the body of metal ore such that the deleterious effects of toxins on the viability and/or function of microorganisms in the microbial lixiviant are lowered. In embodiments, the toxin inactivating microorganisms may have the function of precipitating toxins in non (or less) toxic forms, sequestering toxins and/or metabolizing toxins. Examples of toxins, the levels of which may be lowered by toxin inactivating microorganisms in the present invention include fluoride, arsenic, cobalt, zinc, lead, sulfate, and/or silver. In embodiments, the toxin may be chloride and/or nitrate and the toxin inactivating microorganism may only partially lower the levels of chloride and/or nitrate, and optionally not lower most or all of the chloride and/or nitrate present.

Additionally or alternatively, one, some or all of the strains of microorganisms present in the microbial lixiviant may be toxin resistant, i.e. their viability and/or function is not significantly impacted by the presence of one or more of the toxins described herein.

In certain embodiments, one, some or all of the strains of microorganisms present in the microbial lixiviant may be capable of lowering and/or maintaining a low oxidation reduction potential (ORP) (for example about 500 to about 1000, about 500 to about 900 or about 600 to about 775 mV SHE) in the lixiviant.

In embodiments of the present application, the populations of microorganisms comprised in the microbial lixiviant and/or one or more of the microorganism populations comprised therein may be found together in nature. In alternative embodiments, one or more of the populations of microorganisms comprised in the microbial lixiviant may be artificially combined, i.e. the microbial lixiviant may comprise a rationally designed consortium of microorganisms.

In embodiments in which the microbial lixiviant comprises a rationally designed consortium of microorganisms, the process may comprise steps of providing a sample of the metal ore comprising microorganisms, identifying one or more populations of said microorganisms exhibiting target metabolic function, and isolating said one or more populations of microorganisms and providing said one or more populations of microorganisms in a microbial lixiviant which may then be employed in the process of the present application.

In such embodiments, the step of identifying one or more populations of microorganisms exhibiting target metabolic function may comprise culturing the sample of metal ore and identifying one or more populations of microorganisms which generate heat. The generation of heat may be measured using any technique known to those skilled in the art, for example microcalorimetry.

The microorganisms comprised in the sample of metal ore may be naturally comprised within that sample. Alternatively, the microorganisms may be manipulated, for example by removing certain populations of microorganisms naturally comprised in the sample of metal ore and/or adding populations of microorganisms to the sample of metal ore, e.g. to investigate their metabolic function.

In embodiments in which the step of identifying one or more populations of microorganisms exhibiting target metabolic function comprises culturing the sample of metal ore, this step may be conducted in the presence of one or more contaminants, for example contaminants commonly observed in mining process waters (e.g. released from metal ores during leaching processes, or comprised within lixiviants or other additives to aid and increase metal extraction and recovery), such as halides, arsenic, zinc, cobalt, thiourea, surfactants, nitrate, sulfate or combinations thereof.

As explained above, a critical step of the process of the present invention is adjustment of one or more process variables in response to a measured parameter. By carefully controlling the process variables, the leaching efficiency and/or functional lifespan of the microbial lixiviant can be maximized.

In embodiments, the process variable which is adjusted may be the composition of microorganisms present in the microbial lixiviant. For example, if the measured parameter is indicative of a shift in the microbial composition such that the relative abundance of one or more particular populations of microorganisms is altered to a sub-optimal level (e.g. if that population is depleted meaning that it will have reduced function, if that population is increased meaning that it will outcompete alternative populations of microorganisms which play a critical role in leaching for resources, or if all populations exhibit an unacceptable reduction in function) then the adjustment of the process variable may be the addition of a population of microorganisms (e.g. to increase the abundance of a population which is depleted and/or to increase the abundance of a population which lowers the levels of a toxin (e.g. organic carbon) that may be responsible for a declining population) or the addition of a plurality of populations of microorganisms to lower the relative abundance of another population of microorganisms which is elevated). In embodiments, for example where measurement of the parameter is indicative of a widespread or complete loss of function or viability of the microbes present in the lixiviant, a total replacement of the microbial populations present in the microbial lixiviant may be contacted with the body of metal ore. In such embodiments the microbial populations may be the same or different to those originally contacted with the body of metal ore.

Thus, in embodiments, the process of the present invention comprises contacting the body of metal ore with a second microbial lixiviant, following step iii), comprising one or more populations of microorganisms wherein the microbial composition of the second microbial lixiviant differs from that of the microbial lixiviant contacted with the metal ore in step ii). The microbial composition of the second microbial lixiviant may comprise less populations of microorganisms as compared to the microbial lixiviant used in step ii). Alternatively, the microbial composition of the second microbial lixiviant may comprise more populations of microorganisms as compared to the microbial lixiviant used in step ii). Additionally or alternatively, the microbial composition of the second microbial lixiviant may comprise alternative microbial populations as compared to the microbial lixiviant used in step ii). In embodiments, the microbial composition of the second microbial lixiviant may comprise the same number of microbial populations, and optionally the same microbial populations, as the microbial lixiviant used in step ii) but in different proportions.

In embodiments of the present application, further compositions of microbial lixiviant (“subsequent microbial lixiviant/s”) may be supplied to the body of metal ore, in addition to the microbial lixiviant contacted with the body of metal ore in step ii) and the second microbial lixiviant. The microbial composition of the subsequent microbial lixiviant/s may comprise less populations of microorganisms as compared to the microbial lixiviant used in step ii) and/or the second microbial lixiviant. Alternatively, the microbial composition of the subsequent microbial lixiviant/s may comprise more populations of microorganisms as compared to the microbial lixiviant used in step ii) and/or the second microbial lixiviant. Additionally or alternatively, the microbial composition of the subsequent microbial lixiviant/s may comprise alternative microbial populations as compared to the microbial lixiviant used in step ii) and/or the second microbial lixiviant. In embodiments, the microbial composition of the subsequent microbial lixiviant may comprise the same number of microbial populations, and optionally the same microbial populations, as the microbial lixiviant used in step ii) and/or the second microbial lixiviant but in different proportions.

Where reference is made herein to properties of a microbial lixiviant, these may apply to the microbial lixiviant used in step ii) of the process of the present invention to the second microbial lixiviant and/or to the subsequent microbial lixiviant/s, if used.

Additionally or alternatively, other methods of adjusting the composition of the microorganisms present in the microbial lixiviant may be employed, for example the application of a phage specific to one of the populations of microorganisms or a subset of microorganisms in such a population, the application of an antimicrobial specific to one of the populations of microorganisms or a subset of microorganisms in such a population and/or the application of one or more nutrients which selectively promotes the growth of one or more populations of microorganisms.

Additional process variables which may be adjusted in the methods of the present invention include the rate of application of the microbial lixiviant to the body of metal ore, the temperature at which the metal ore is maintained and/or agitation or other mechanical disruption of the metal ore. Those skilled in the art will recognize that the control of certain process variables may be more readily achievable depending on the way in which the process is conducted. For example, if the body of metal ore is provided in a tank, then this may make control of the temperature at which the process is operated more straightforward. However, all combinations of process variables and types of leaching process are encompassed by the present application.

Conveniently, the microbial lixiviants discussed herein may be cultured and/or prepared at the site at which those lixiviants will be contacted with the body of metal ore. For example, bioreactor apparatus optionally comprising one or more bioreactor tanks, may be provided in the vicinity of the body of metal ore (e.g. within about 10 km, about 5 km, about 2 km, about 1 km, about 500 m, about 200 m, about 100 m, about 50 m, about 20 m or about 10 m). A biomass may be recovered from the bioreactor apparatus. The biomass may be contacted directly with the body of metal ore (in which case the biomass will be the microbial lixiviant (or second microbial lixiviant and/or subsequent microbial lixiviant/s, if used)). Alternatively, the biomass may be fed into a lixiviant stream which is then contacted with the body of metal ore as the microbial lixiviant (or second microbial lixiviant and/or subsequent microbial lixiviant/s, if used). In embodiments, the biomass recovered from the bioreactor apparatus may comprise about 10⁶ to about 10¹³ cells/L of viable microorganisms. In certain embodiments, the biomass recovered from the bioreactor apparatus may comprise about 10⁸ to about 10¹² cells/L of viable microorganisms.

In embodiments of the present application, the process may comprise the step of providing the microbial lixiviant with a nutrient composition. This may be combined within the microbial lixiviant prior to it being contacted with the body of metal ore and/or be contacted with the microbial lixiviant after it the microbial lixiviant is contacted with the metal ore. Such nutrient composition may comprise microbial energy sources, for example raffinate.

In embodiments of the present application, the microbial lixiviant may comprise one or more components employed in conventional leach lixiviants, for example an acid (e.g. sulfuric acid) and/or a source of ferric iron (for example a ferrous salt such as ferrous sulfate which can be converted microbially into ferric iron, or a ferric salt such as ferric sulfate).

In certain embodiments of the present application, the microbial lixiviant is acidic. In such embodiments, the pH may be about 0 to about 5, about 1 to about 4, about 1 to about 3 or about 2 to about 4.

In certain embodiments, the microbial lixiviant may comprise an nutrient source, e.g. ammonium (e.g. ammonium sulfate), phosphate (e.g. potassium phosphate), carbonate (e.g., sodium carbonate), and/or nitrate (e.g. calcium nitrate).

In an embodiment, the microbial lixiviants discussed herein, e.g. the compositions defined herein and/or the microbial lixiviant/s supplied to the body of metal ore (for example, the lixiviant contacted with the body of metal ore in step ii), the second microbial lixiviant and/or the subsequent microbial lixiviant/s) may comprise microorganisms in an amount of at least about 10² cells/mL, at least about 10³ cells/mL, at least about 10⁴ cells/mL, at least about 10⁵ cells/mL, at least about 10⁶ cells/mL, at least about 10⁷ cells/mL or at least about 10⁸ cells/mL.

In alternative embodiments, the microbial lixiviants discussed herein, e.g. the compositions defined herein and/or the microbial lixiviant/s supplied to the body of metal ore (for example, the lixiviant contacted with the body of metal ore in step ii), the second microbial lixiviant and/or the subsequent microbial lixiviant/s) may comprise microorganisms in an amount of about 10¹⁰ cells/mL or lower, about 10⁸ cells/mL or lower, about 10⁷ cells/mL or lower, about 10⁶ cells/mL or lower, about 10⁵ cells/mL or lower, or about 10⁴ cells/mL or lower.

An advantage of the present invention is that, unlike conventional bioleaching processes, the monitoring of microbial function and/or viability through the measurement of parameter/s permits a comprehensive understanding of the abundance and/or function of specific microbial populations. This in turn enables effective leaching efficiency to be maintained through the periodic provision of smaller numbers of microorganisms to the body of metal ore, in contrast to the prior art approach of simply supplying ore with vast volumes of microbes in the hope of this translating into leaching efficiency. Thus, in embodiments of the present application, the microbial lixiviant discussed herein, e.g. the compositions defined herein and/or the microbial lixiviant/s supplied to the body of metal (for example, the lixiviant contacted with the body of metal ore in step ii), the second microbial lixiviant and/or the subsequent microbial lixiviant/s) ore may comprise microorganisms in an amount from about 10² cells/mL to about 10⁸ cells/ml. In embodiments, the microbial lixiviant discussed herein may comprise microorganisms in an amount from about 10² cells/mL to about 10⁷ cells/mL. In embodiments the microbial lixiviant discussed herein may comprise microorganisms in an amount of from about 10³ cells/mL to about 10⁶ cells/mL.

In some embodiments of the present application, the microbial lixiviant discussed herein, e.g. the compositions defined herein and/or the microbial lixiviant/s supplied to the body of metal (for example, the lixiviant contacted with the body of metal ore in step ii), the second microbial lixiviant and/or the subsequent microbial lixiviant/s) ore may consist of essentially 2 or more, 3 or more, 4 or more, 5 or more, 6 or more, 7 or more, 8 or more, 9 or more, 10 or more, 11 or more, 12 or more, 13 or more, 14 or more, 15 or more or 16 or more strains of microorganisms. Additionally or alternatively, the microbial lixiviant discussed herein, e.g. the compositions defined herein and/or the microbial lixiviant/s supplied to the body of metal ore (for example, the lixiviant contacted with the body of metal ore in step ii), the second microbial lixiviant and/or the subsequent microbial lixiviant/s) may consist essentially of 50 or fewer, 40 or fewer, 30 or fewer, or 20 or fewer, 10 or fewer, 8 or fewer, 7 or fewer, 6 or fewer or 5 or fewer strains of microorganism. In certain embodiments, the microbial lixiviant discussed herein may consist essentially of 2 to 50 strains of microorganisms. In an embodiment the microbial lixiviant discussed herein may consist essentially of 2 to 30 strains of microorganisms. In a further embodiment, the microbial lixiviant discussed herein may consist of essentially 2 to 20 strains of microorganisms. The microbial lixiviant discussed herein may consist essentially of 2 to 10 strains of microorganisms. In a specific embodiment, the microbial lixiviant discussed herein may consist essentially of 2 to 8 strains of microorganism. In further embodiments, the microbial lixiviant discussed herein may consist essentially of 2 to 6 strains of microorganism or 2 to 5 strains of microorganism.

In embodiments of the present application, the microbial lixiviant discussed herein, e.g. the compositions defined herein and/or the microbial lixiviant/s supplied to the body of metal (for example, the lixiviant contacted with the body of metal ore in step ii), the second microbial lixiviant and/or the subsequent microbial lixiviant/s) ore may comprise 2 or more, 3 or more, 4 or more, 5 or more, 6 or more, 7 or more, 8 or more, 9 or more, 10 or more, 11 or more, 12 or more, 13 or more, 14 or more, 15 or more or 16 or more, or 2 to 10, 2 to 8, 2 to 6, 2 to 5 or 2 to 4 strains of microorganisms having an abundance of at least 10⁴ cells/mL.

In embodiments of the present application, the microbial lixiviant discussed herein, e.g. the compositions defined herein and/or the microbial lixiviant/s supplied to the body of metal (for example, the lixiviant contacted with the body of metal ore in step ii), the second microbial lixiviant and/or the subsequent microbial lixiviant/s) ore may comprise 2 or more, 3 or more, 4 or more, 5 or more, 6 or more, 7 or more, 8 or more, 9 or more, 10 or more, 11 or more, 12 or more, 13 or more, 14 or more, 15 or more or 16 or more, or 2 to 10, 2 to 8, 2 to 6, 2 to 5 or 2 to 4 strains of microorganisms having an abundance of at least 10⁵ cells/mL.

In embodiments of the present application, the microbial lixiviant discussed herein, e.g. the compositions defined herein and/or the microbial lixiviant/s supplied to the body of metal (for example, the lixiviant contacted with the body of metal ore in step ii), the second microbial lixiviant and/or the subsequent microbial lixiviant/s) ore may comprise 2 or more, 3 or more, 4 or more, 5 or more, 6 or more, 7 or more, 8 or more, 9 or more, 10 or more, 11 or more, 12 or more, 13 or more, 14 or more, 15 or more or 16 or more, or 2 to 10, 2 to 8, 2 to 6, 2 to 5 or 2 to 4 strains of microorganisms having an abundance of at least 10⁶ cells/mL.

As used herein, the term “population” is used to describe a group of strains of microorganisms exhibiting common function. Examples of populations of microorganisms which may be present in microbial lixiviants as disclosed herein include iron oxidizers, sulfur oxidizers, organic carbon degraders and/or toxin inactivating microorganisms. For the avoidance of doubt, a single strain having varied functionality such that it could be considered to belong to more than one of the populations discussed herein, that strain shall nevertheless be considered to belong to only a single population. For example, if a composition comprises a single strain which has iron oxidizing functionality and sulfur oxidizing functionality, then for the purposes of the present disclosure, that composition will be considered to comprise one population, and not two populations.

The compositions disclosed herein may comprise one or more populations, two or more populations, three or more populations or four or more populations selected from iron oxidizers, sulfur oxidizers, organic carbon degraders and/or toxin inactivating microorganisms. In certain embodiments, the compositions disclosed herein may comprise 1 to 4, 1 to 3, 1 to 2, 2 to 3, 2 to 4 or 3 to 4 of said populations.

In embodiments, the microorganisms within a population may all belong to the same species, the same genus, the same family, the same order, the same class, the same phylum, the same kingdom or the same domain. Alternatively, the microorganisms within a population may belong to 1 or more, 2 or more, 3 or more, 4 or more, or 5 or more, genera of microorganism. In embodiments, the microorganisms within a population may belong to 1 or more, 2 or more, 3 or more, 4 or more, or 5 or more species of microorganism.

As used herein, to describe the bacterial population comprised within the composition of the present application, the term ‘consisting essentially of’ and ‘consisting of’ is used to characterize the composition as excluding additional bacterial strains or species, or comprising only de minimis or biologically irrelevant amounts of other bacterial strains or species.

In some embodiments of the present application, a population of microorganisms may consist of essentially 1 or more, 2 or more, 3 or more, 4 or more, 5 or more, 6 or more, 7 or more, 8 or more, 9 or more or 10 or more strains of microorganisms. Additionally or alternatively, a population of microorganisms may consist essentially of 50 or fewer, 40 or fewer, 30 or fewer, 20 or fewer, 10 or fewer, 8 or fewer, 6 or fewer, 5 or fewer, 4 or fewer, 3 or fewer or 2 or fewer strains of microorganism. In certain embodiments, a microorganism population may consist essentially of 1 to 50 strains of microorganisms. In an embodiment, a microorganism population may consist essentially of 1 to 30 strains of microorganisms. In a further embodiment, a microorganism population may consist of essentially 1 to 20 strains of microorganisms. A microorganism population may consist essentially of 1 to 10 strains of microorganisms. In a specific embodiment, a microorganism population may consist essentially of 1 to 5 strains of microorganism. In further embodiments, a microorganism population may consist essentially of 1 to 3 strains of microorganism.

In an embodiment, one, some or all of the populations comprised in the microbial lixiviants discussed herein, e.g. the compositions defined herein and/or the microbial lixiviant/s supplied to the body of metal ore (for example, the lixiviant contacted with the body of metal ore in step ii), the second microbial lixiviant and/or the subsequent microbial lixiviant/s) may comprise microorganisms in an amount of at least about 10² cells/mL, at least about 10³ cells/mL, at least about 10⁴ cells/mL, at least about 10⁵ cells/mL, at least about 10⁶ cells/mL, at least about 10⁷ cells/mL or at least about 10⁸ cells/mL.

In embodiments of the present application, one, some or all of the populations comprised in the microbial lixiviant discussed herein, e.g. the compositions defined herein and/or the microbial lixiviant/s supplied to the body of metal (for example, the lixiviant contacted with the body of metal ore in step ii), the second microbial lixiviant and/or the subsequent microbial lixiviant/s) ore may comprise 1 or more, 2 or more, 3 or more, 4 or more, 5 or more, 6 or more, 7 or more, 8 or more, 9 or more, 10 or more, 11 or more, 12 or more, 13 or more, 14 or more, 15 or more or 16 or more, or 2 to 10, 2 to 8, 2 to 6, 2 to 5 or 2 to 4 strains of microorganisms having an abundance of at least 10⁴ cells/mL.

In embodiments of the present application, one, some or all of the populations comprised in the microbial lixiviant discussed herein, e.g. the compositions defined herein and/or the microbial lixiviant/s supplied to the body of metal (for example, the lixiviant contacted with the body of metal ore in step ii), the second microbial lixiviant and/or the subsequent microbial lixiviant/s) ore may comprise 1 or more, 2 or more, 3 or more, 4 or more, 5 or more, 6 or more, 7 or more, 8 or more, 9 or more, 10 or more, 11 or more, 12 or more, 13 or more, 14 or more, 15 or more or 16 or more, or 2 to 10, 2 to 8, 2 to 6, 2 to 5 or 2 to 4 strains of microorganisms having an abundance of at least 10³ cells/mL.

In embodiments of the present application, one, some or all of the populations comprised in the microbial lixiviant discussed herein, e.g. the compositions defined herein and/or the microbial lixiviant/s supplied to the body of metal (for example, the lixiviant contacted with the body of metal ore in step ii), the second microbial lixiviant and/or the subsequent microbial lixiviant/s) ore may comprise 1 or more, 2 or more, 3 or more, 4 or more, 5 or more, 6 or more, 7 or more, 8 or more, 9 or more, 10 or more, 11 or more, 12 or more, 13 or more, 14 or more, 15 or more or 16 or more, or 2 to 10, 2 to 8, 2 to 6, 2 to 5 or 2 to 4 strains of microorganisms having an abundance of at least 10⁶ cells/mL.

In embodiments of the present application, the microbial lixiviant discussed herein may be free of genetically modified microorganisms. In alternative embodiments, the microbial lixiviant discussed herein may principally or exclusively comprise microorganisms which are not genetically modified microorganisms. In such embodiments, at least 50%, at least 60%, at least 70%, at least 80%, at least 90%, at least 95%, at least 98%, at least 99% or at least 99.5% of the microorganisms present in the microbial lixiviant are not genetically modified organisms.

In other embodiments of the present application, the microorganisms present in the microbial lixiviant may principally or exclusively comprise organisms which are genetically modified. In such embodiments, at least 50%, at least 60%, at least 70%, at least 80%, at least 90%, at least 95%, at least 98%, at least 99% or at least 99.5% of the microorganisms present in the microbial lixiviant are genetically modified organisms.

In an embodiment, microbial lixiviant may be continuously supplied to the body of metal ore. As used herein “continuous” or “continuously” is not to be interpreted in a strictly literal sense but also encompasses situations in which the supply of microbial lixiviant may be temporarily paused, e.g. to permit the change or replacement of apparatus or sources of lixiviant to be contacted with the body of metal ore. The continuous supply of microbial lixiviant to a body of metal ore may mean that the body of metal ore is continuously supplemented with microbial lixiviant comprising a functioning and effective population or populations of microorganisms for the duration of the bioleaching process.

Continuously supplying a microbial lixiviant comprising functionally diverse microorganisms can buffer chemical imbalances and prevent the accumulation of contaminants such as sulfur or organic carbon rapidly and locally before they become a factor that impacts leaching efficiency. In certain embodiments, the microbial composition and/or rate of supply of microbial lixiviant to the body of metal ore may be controlled such that the adjustment of the microbial populations in the microbial lixiviant and/or the body of metal ore is gradual. In normal operation of the process, such gradual control of the microbial composition is preferable as this permits leaching activity to continue without interruptions caused by populations which are either not present or which are present but at low levels being added and needing to establish an effective population. However, in other situations, for example, when the measured parameters are indicative of a rapid and/or significant loss in microbial viability and/or function, then the one or more microbial populations may need to be contacted with the body of metal ore, via the lixiviant, in high volumes and/or at high flow rates.

In an embodiment, microbial lixiviant is added to the body of metal ore at a rate of about 10 L/h per m² of surface of the body of metal ore.

The process of the present invention allows for the efficient recovery of metals from metal ores. The metal of interest may be present in the ore at a level of 5% or less by weight. However, in embodiments, the metal of interest may be present in the ore at lower levels, for example at a level of 4% or less, 3% or less, 2% or less, or 1% or less.

In certain embodiments, the metal of interest may be present in the ore at a level of about 5000 ppm or less, at a level of about 2000 ppm or less, at a level of about 1000 ppm or less, about 500 ppm or less, about 200 ppm or less, about 100 ppm or less or about 50 ppm or less. Additionally or alternatively, the metal of interest may be present in the ore at a level of at least about 1 ppm, at least about 2 ppm, at least about 5 ppm, at least about 10 ppm, at least about 20 ppm, at least about 50 ppm, or at least about 100 ppm.

In some embodiments of the present invention, the metal ore will be contacted with microbial lixiviant about every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days. In some embodiments of the present application, the metal ore will be contacted with microbial lixiviant about every 1-30, 1-25, 1-20, 1-15, 1-10, or 1-5 days.

Following contact of the body of metal ore with the microbial lixiviant (and second microbial lixiviant and/or subsequent microbial lixiviant/s, if used), metal will be released from the metal ore and thus, in embodiments of the present application, the process further comprises collecting a stream from the body of metal ore comprising the metal. This may be achieved through using techniques familiar to one skilled in the art.

In embodiments, a lixiviant efflux stream may be collected from the body of metal ore and this may be recycled to the body of metal ore. Prior to being recycled, the microbial composition, diversity and/or metabolic function of the microbial lixiviant may be assessed. Additionally or alternatively, the lixiviant efflux stream may be treated, e.g. by the addition of microorganisms belonging to one or more microorganism populations.

The invention will now be illustrated by the examples which follow.

### EXAMPLES

The following examples describe selected aspects and embodiments of the present teachings. These examples are intended for the purposes of illustration and should not be construed to limit the cope of the present application.

### Example 1

A 1:1 mix of copper concentrate comprising mainly chalcopyrite with a 26.8% copper content and 27.4% iron content, and raw copper ore with 0.6% copper, 10% iron, was used.

1 g of the ore mix and 100 ml of leaching solution (3.0 g/L (NH₄)₂SO₄, 0.5 g/L K₂HPO₄, 0.5 g/L MgSO₄×7H₂O, 0.1 g/L KCl and 0.1 g/L Ca(NO₃)₂) adjusted to a pH of 1.8 with sulfuric acid were mixed and poured into a 250 ml stirred flask. This mixture was stirred at 35° C. A biomass comprising 10⁶ cells/mL was added to the mixture. The biomass comprised the following strains: Acidithiobacillus thiooxidans (sulfur oxidizer), Ferroplasma acidophilum (iron oxidizer, organic carbon degrader), Leptospirillum ferrooxidans (iron oxidizer), Sulfobacillus thermosulfidooxidans (iron oxidizer, sulfur oxidizer, organic carbon degrader).

As a comparative example, an identical ore/lixiviant slurry was prepared but was not inoculated with microorganisms and was kept sterile.

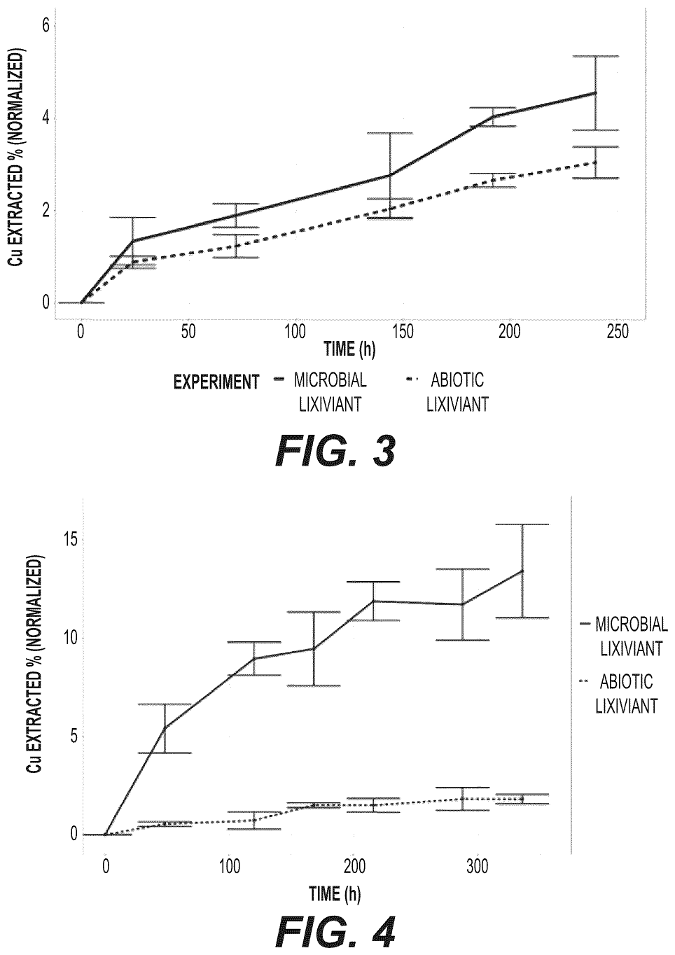

The copper recovered was measured using atomic absorption spectrometry (MS) and the results are shown in FIG. 3.

The experiment was repeated using identical conditions except that a different biomass was used comprising 10⁶ cells/mL of the following strains: Acidithiobacillus ferrooxidans (sulfur oxidizer, iron oxidizer) and Leptospirillum ferriphilum (iron oxidizer).

The copper recovered was measured using atomic absorption spectrometry (MS) and the results are shown in FIG. 4.

The data shown in FIGS. 3 and 4 demonstrate that the copper leaching efficiency of a lixiviant comprising a rationally designed consortium of microorganisms belonging to different populations (iron oxidizers, sulfur oxidizers and organic carbon degraders in the case of the results shown in FIG. 3 and iron oxidizers and sulfur oxidizers in the case of the results shown in FIG. 4) are greater than a conventional abiotic lixiviant.

### Example 2

In a modelled example, two 50 kg samples of copper ore having an average diameter of 0.5 to 25 mm and representative in composition to ore commonly found in industrial mining operations in Arizona was added to a leach column and irrigated with a synthetic raffinate at a flow of 10 liters per in² per hour. The synthetic raffinate was continuously supplemented with a mix of iron and sulfur-oxidizing, mesophilic, autotrophic and organic carbon-sensitive, acidophilic bacteria such as typically found in metal sulfide heap leaching to a concentration of 10⁴ cells/ml in the raffinate (Inoculant A). Copper contents in solution at the bottom of the leach column were measured using atomic absorption spectrometry (MS), extracting filtered samples in a micro-porous material (5 μm pore diameter).

Simulated organic carbon content in the raffinate was gradually increased by addition of YE until a concentration of 0.02% was reached on day 40, at which point raffinate inoculation of one column was switched to a mix of iron and sulfur-oxidizing, mesophilic, organic carbon tolerant and degrading, acidophilic bacteria at the same concentration (Inoculant B) while for the other column the original inoculant composition was retained. Incubation continued until day 100.

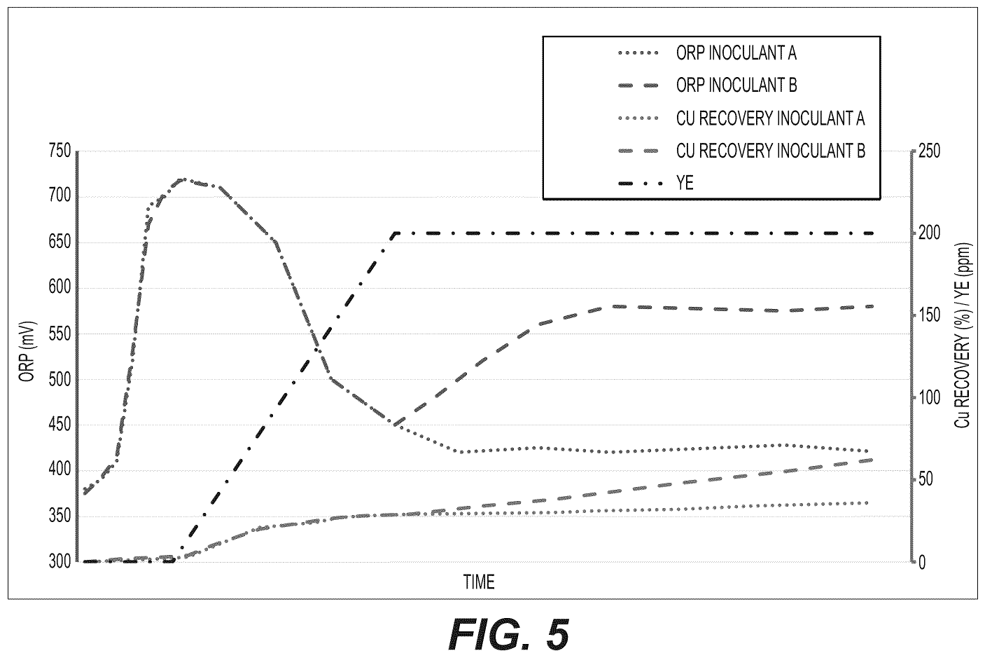

The modelled data is shown in FIG. 5 and shows a decline in the rate of copper extraction with increasing concentration of organic carbon when using Inoculant A and its recovery when replacing the inoculant with Inoculant B.

This modelled data demonstrates that, by monitoring variables in a body of metal ore such as organic carbon content, the microbial composition in the lixiviant added to that body can be tailored to maximize leaching efficiency.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

1. A process comprising the steps of:
i) providing a body of metal ore,
ii) contacting the body of metal ore with a microbial lixiviant comprising one or more populations of microorganisms,
iii) measuring at least one parameter indicative of function of the one or more populations of microorganisms, and
iv) adjusting one or more process variables in response to the measured parameter.

2. The process of claim 1, wherein the body of metal ore is a heap or wherein the body of metal ore is provided in a tank.

3. The process of claim 1, wherein the microbial lixiviant is supplied to the body of metal ore at a rate of about 10 L/h per m² of surface of the body of metal ore.

4. The process of claim 1, wherein the at least one parameter comprises a microbial composition and/or a microbial diversity of the microbial lixiviant.

5. The process of claim 1, wherein the measuring step comprises taking a baseline measurement of the at least one parameter and a subsequent measurement and comparing the measurements.

6. The process of claim 1, wherein the one or more process variables comprise a microbial composition of the microbial lixiviant in contact with the body of ore and/or a microbial composition of the microbial lixiviant comprised within the body of ore.

7. The process of claim 1, further comprising the step of contacting the body of metal ore with a second microbial lixiviant having a microorganism composition different from the microbial lixiviant contacted with the metal ore in step ii).

8. The process of claim 1, wherein the microbial lixiviant is continuously supplied to the body of metal ore.

9. The process of claim 1, wherein the microbial lixiviant is intermittently supplied to the body of metal ore.

10. The process of claim 1, wherein the metal content of the metal ore prior to being contacted with the microbial lixiviant is about 5000 ppm or less.

11. The process of claim 1, wherein the microbial lixiviant comprises 2 or more populations of microorganisms.

12. The process of claim 1, wherein the microbial lixiviant comprises at least one, population of microorganisms selected from the group consisting of iron oxidizers, sulfur oxidizers, organic carbon degraders and/or toxin inactivating microorganisms.

13. A microbial lixiviant composition for use in a bioleaching process, comprising a plurality of strains of microorganisms forming one or more populations of microorganisms selected from the group consisting of: iron oxidizers, sulfur oxidizers, organic carbon degraders and toxin inactivating microorganisms.

14. The microbial lixiviant composition of claim 13, wherein the microbial lixiviant composition comprises microorganisms not found together in nature.

15. The microbial lixiviant composition of claim 13, wherein the microbial lixiviant composition comprises one or more nutrients.

16. The process of claim 1, wherein the microbial lixiviant comprises microorganisms in a range of about 10² cells/mL to about 10⁸ cells/ml.

17. The process of claim 1, wherein the microbial lixiviant comprises one or more microorganism populations selected from the group consisting of:
iron-oxidizer microorganism population, optionally comprising microorganisms belonging to one or more of the following genera: Acidocella, Acidimicrobium, Acidiphilium, Acidiplasma, Acidithiobacillus, Acidithiomicrobium, Acidithrix, Ferrithrix, Ferroplasma, Ferrovum, Halomonas, Leptospirillum, Metallosphaera, Mycobacterium, Sulfobacillus, Sulfolobus and Thermogymnomonas,
sulfur oxidizer microorganism population, optionally comprising microorganisms belonging to one or more of the following genera: Acidianus, Acidicaldus, Acidithiobacillus, Metallosphaera and Sulfolobus,
organic carbon degrader microorganism population, optionally comprising microorganisms belonging to one or more of the following genera: Acidicaldus, Acidianus, Acidocella, Acidimicrobium, Acidiphilium, Acidiplasma, Acidimicrobium, Alicyclobacillus, Ferrithrix, Ferroplasma, Ferrovum, Metallosphaera, Picrophilus, Sulfobacillus, Sulfolobus and Thermoplasma,
and
toxin-inactivating microorganism population.

18. The process of claim 1, wherein the one or more of the populations of microorganisms in the microbial lixiviant consist of one or more strains of microorganisms.

19. The process of claim 1, wherein one or more of the populations of microorganisms consist of 10 or fewer strains of microorganisms.

20. The process of claim 1, wherein the microorganisms present in the microbial lixiviant are not genetically modified microorganisms.
